(19) United States
(12) Patent Application Publication
(10) Pub. No.: US 2024/0027449 A1
(43) Pub. Date: Jan. 25, 2024

(54) DIAGNOSTIC METHOD AND SYSTEM WITH IMPROVED SENSITIVITY

(71) Applicant: NATIONAL TAIPEI UNIVERSITY OF TECHNOLOGY, Taipei (TW)

(72) Inventor: Shao-Yi Hou, Taipei (TW)

(73) Assignee: NATIONAL TAIPEI UNIVERSITY OF TECHNOLOGY, Taipei (TW)

(21) Appl. No.: 17/871,343

(22) Filed: Jul. 22, 2022

(51) Int. Cl. G01N 33/569; G01N 33/543; G01N 33/554

(52) U.S. Cl. CPC G01N 33/56983; G01N 33/54388; G01N 33/554

(57) ABSTRACT

The present invention provides an improvement method and a system for decreasing the limit of detection of the presence of an analyte in a sample. In particular, the present invention provides an article or a system, such as a diagnostic kit, for detecting the presence of an analyte in a sample, comprising (i) a microsphere coated with an affinity ligand, or a spore or bacterium expressing one or more proteins on the surface thereof, (ii) a signal-producing substances, and (iii) a binding agent, wherein the signal-producing substance is conjugated with the binding agent and an antibody specific to the affinity ligand on the microsphere or the protein expressed by the spore or bacterium, wherein the signal-producing substances are conjugated to the microsphere, spore or bacterium through the binding of the antibodies specific to the affinity ligand or the protein.

### FIELD OF THE INVENTION

The present invention relates to articles, methods and systems for detecting the presence of an analyte in a sample with improved sensitivity, particularly using microspheres, spores or bacteria as detecting agents.

### BACKGROUND OF THE INVENTION

Lateral flow assay (LFA) is a paper-based platform for the detection and quantification of analytes in complex mixtures, where the sample is placed on a test device and the results are typically displayed within 5-30 minutes. Low development costs and ease of production of LFA have resulted in the expansion of its applications to multiple fields in which rapid tests are required. LFA-based tests are widely used in hospitals, physicians' offices and clinical laboratories for qualitative and quantitative detection of specific antigens and antibodies. A variety of biological samples can be tested using LFAs, including urine, saliva, sweat, serum, plasma, whole blood and other fluids. Further industries in which LFA-based tests are employed include veterinary medicine, quality control, product safety in food production, and environmental health and safety. In these areas of utilization, rapid tests are used to screen for animal diseases, pathogens, chemicals, toxins and water pollutants, among others.

The principle behind LFA is simple: a liquid sample (or its extract) containing the analyte of interest moves without the assistance of external forces (capillary action) through various zones of polymeric strips, on which molecules that can interact with the analyte are attached. A typical lateral flow test strip consists of overlapping membranes that are mounted on a backing card for better stability and handling. The sample is applied at one end of the strip, on the adsorbent sample pad, which is impregnated with buffer salts and surfactants that make the sample suitable for interaction with the detection system. The sample pad ensures that the analyte present in the sample will be capable of binding to the capture reagents of conjugates and to the membrane. The treated sample migrates through the conjugate release pad, which contains antibodies that are specific to the target analyte and are conjugated to colored or fluorescent particles-most commonly colloidal gold nanoparticles (AuNPs) and latex microspheres. The sample, together with the conjugated antibody bound to the target analyte, migrates along the strip into the detection zone. This is a porous membrane (usually composed of nitrocellulose) with specific biological components (mostly antibodies or antigens) immobilized in test and control lines on the membrane. Their role is to react with the analyte bound to the conjugated antibody. Recognition of the analyte in a sample results in an appropriate response on the test line, while a response on the control line indicates the proper liquid flow through the strip. The read-out, represented by the lines appearing with different intensities, can be assessed by eye or using a dedicated reader. The liquid flows across the device because of the capillary force of the strip material and, to maintain this movement, an absorption pad is attached at the end of the strip. The role of the absorption pad is to wick the excess reagents and prevent backflow of the liquid. FIG. 1 illustrates an example of such conventional LFA system in which N-protein represents the analyte to be detected.

In the common practice, colloidal gold or colored latex beads conjugated with antibodies are generally used as labeling in lateral flow immunochromatographic assays (LFIAs). Even with a high specificity of >90% in clinical diagnosis, the immunochromatography method is notoriously known for its overall low sensitivity of approximately 60%. For example, in regard to the low sensitivity, the samples required for detection of influenza virus are limited by high-concentration samples acquired through throat swabs or blood samples, which is inconvenient and may produce physical discomfort for patients.

Moreover, the conventional labels have drawbacks such as slow detection time, difficulty of detecting antigens at low concentration, and lack of colorability. Specifically, although the colloidal gold nanoparticles (e.g., Prorast™-Flu) produce a weak, single type of color, they have a deep color and a small surface area; however, the colored latex beads (e.g., QuickNavi™-Flu) fail to produce a deep color, whereas they have a large surface area. Obviously, there is a desperate need for progress with respect to the labels utilized in lateral flow immunochromatographic assays. As development of the labels continues, the higher sensitivity will significantly add value to clinical diagnoses.

Surface display requires expression of a target protein on the surface of the cell membrane of living cells through genetic engineering techniques. For the display to be successful, the target protein needs to be fused with an anchor protein (Van Bloois et al., “Decorating microbes: surface display of proteins on Escherichia coli.” Trends Biotechnol; 29: 79-86, 2011) in order to display translocated-incompatible and multimeric proteins (Kim and Schumann, “Display of proteins on Bacillus subtilis endospores.” Cell Mol Life Sci; 66: 3127-3136, 2009). The first surface display system was developed by George P. Smith et al. in 1985, wherein antibodies were expressed on the surface of phages using filamentous bacteriophage M13. Said system provides a new technique for antigen production (“Filamentous fusion phage: Novel expression vectors that display cloned antigens on the virion surface.” Science, 228 (4705), pp. 1315-1317, 1985). The surface display technique has since been applied to other organisms such as bacteria, yeasts and spores.

Extensive studies have been performed with respect to surface display systems in Gram-negative bacteria. Among those, Escherichia coli (E. coli) has been widely studied, and found to display heterologous proteins on the cell surface (Francisco et al., “Transport and anchoring of 8-lactamase to the external surface of Escherichia coli.” Proc. Natl. Acad. Sci. USA, Vol. 89, pp. 2713-2717, April 1992; Georgiou et al., “Display of pMactamase on the Escherichia coli surface: outer membrane phenotypes conferred by Lpp′-OmpA′-β-lactamase fusions.” Protein Engineering Vol. 9, No. 2, pp. 239-247, 1996). Professor George Georgiou's group constructed a fusion protein containing (i) the signal sequence and first nine N-terminal amino acids of the mature lipoprotein, an outer membrane protein of E. coli, (ii) the amino acids 46-159 of OmpA, another outer membrane protein of E. coli, and (iii) the complete β-lactamase. It was demonstrated that this fusion protein is expressed outside of E. coli and exhibits β-lactamase activity (Francisco et al., 1992, Georgiou et al., 1996). Surface display of E. coli has a variety of different applications, such as whole-cell biocatalysts, biosorbents, peptide screening, vaccine production, and antibody production (Nguyen and Schumann, “Use of IPTG-inducible promoters for anchoring recombinant proteins on the Bacillus subtilis spore surface.” Protein Expression and Purification, 95, pp. 67-76, 2014).

Compared with systems in other organisms, surface display on spores has advantages in terms of high stability, easy purification and recovery, and the ability to express large molecules. Due to intracellular production, spores are advantageous compared with non-spore producers in view of the fact that heterologously anchored proteins cannot cross any membrane. Moreover, because of a rigid spore coat, proteins and enzymes displayed on spores become resistant to harsh conditions such as high temperature, chemicals and radiation during industrial procedures; they can also be stored for a long time at room temperature (Kim and Schumann, 2009).

Bacillus subtilis is an aerobic, Gram-positive bacterium. It widely exists in soil, lakes, oceans, animals and plants. While Bacillus subtilis has been found in human intestines, it is non-pathogenic. The most commonly used strains of Bacillus subtilis in laboratories are strains 168, PY79, W23 and NCIB3610, of which the genome of strain 168 has been completely sequenced. The size of Bacillus subtilis is about 0.7 to 0.8×3 μm; it has no capsule, has flagella all over the surface, and is mobile.

Under extreme conditions, Bacillus subtilis has the ability to enter sporulation and survive for a long time under harsh environmental conditions. At first, the cells divide to produce smaller prespores and larger mother cells, which are separated by a membrane in between. In the next stage, the mother cells phagocytose the prespores, and the surface of the prespores produces a peptidoglycan cortex and spore coat. The prespores are then released from the mother cells after maturation (Al-Hinai et al., “The Clostridium Sporulation Programs: Diversity and Preservation of Endospore Differentiation.” Microbiology and Molecular Biology Reviews, 79 (1), pp. 19-37, 2015). Spores are of a complex multi-layer structure, which consists mainly of four layers: the innermost core containing the important genetic material, DNA, which is surrounded by the inner membrane; the peptidoglycan cortex with peptidoglycan as the main component; the spore coat composed of several layers of proteins including the basement layer, inner coat, outer coat, and crust; and the exosporium (Setlow, “Germination of Spores of Bacillus Species: What We Know and Do Not Know.” Journal of Bacteriology, 196 (7), pp. 1297-1305, 2014; Henriques et al., Functional architecture and assembly of the spore coat; in Ricca E, Henriques AO, Cutting SM (eds): Bacterial Spore Formers: Probiotics and Emerging Applications. London, Horizon Science Press, pp. 34-52, 2004).

Bacillus subtilis spores contain at least 70 different spore coat proteins, including CotA, CotB, CotC, CotD, CotE, CotF, CotG, CotH CotJA, CotJC, CotM, CotS, CotSA, CotT, CotX, CotY, CotZ, SpoIVA, SpoVID, YabG, and YrbA (McKenney et al., “The Bacillus subtilis endospore: assembly and functions of the multilayered coat” (2013) Nature Reviews Microbiology, 11, pp. 33-44; Takamatsu and Watabe, Assembly and genetics of spore protective structures. Cell Mol Life Sci 2002; 59: 434-444), but the most preferred anchored proteins are the outer coat proteins.

A number of factors affect the efficiency of Bacillus subtilis spore surface display systems, including anchor proteins, target proteins, linkers, expression vectors and other experimental parameters. In recent years, many anchor proteins have been reported for use in Bacillus subtilis spore surface display, including CotB, CotC, CotG, CotZ, CotX, CotY, CotA, OxdD, CotE, CotZ, CgeA and other coat proteins. Of these, CotB, CotC and CotG have been studied in depth. CotB was the first spore coat protein to be used in spore surface display technology, and different lengths of CotB have worked as anchor proteins to successfully locate exogenous proteins on the spore surface (Isticato et al., “Surface display of recombinant proteins on Bacillus subtilis spores.” J. Bacteriol. 183: 6294-6301, 2001). Linker peptides can form stable helical structures to solve the problem of having a rigid structure between the anchor protein and target protein. Substantial research has shown that inclusion of flexible linker peptides in constructing a recombinant vector is an effective way to regulate the function of fusion enzymes. Fusion of exogenous and anchor proteins can be achieved by involving the N-terminal, C-terminal and sandwich structures of the proteins. The fusion method is determined through the direction of anchoring during the process of sporulation, which locates a target protein on the spore surface being expressed with anchor proteins. Bacillus subtilis spore surface display can be conducted by recombinant and non-recombinant fusion approaches (Isticato, “Spore Surface Display.” Microbiol. Spectr 2(5), 2014). The method of recombination is mostly based on fusion of the genes encoding the foreign and anchor proteins, using either integrated or episomal plasmids. Along with the induction of the spore formation process, foreign proteins are successfully displayed on the spore surface without affecting the structure and function of the spores.

Whole cell-based biosensors are inexpensive and easy to operate, and serve as an alternative for fast screening (Bereza-Malcolm et al., 2015; Mehta et al., 2016). In said method, a synthetic gene is incorporated into a cell, wherein a specific compound is targeted, and an easily detectable signal is produced (Liu et al., “Cell-based biosensors and their application in biomedicine.” Chem. Rev., 114, 6423-6461, 2014; Tian et al., “Construction and comparison of yeast whole-cell biosensors regulated by two RAD54 promoters capable of detecting genotoxic compounds.” Toxicol. Mech. Methods, 27, pp. 115-120, 2017). For example, chemical activated luciferase gene expression (calux) can be used to detect specific chemicals (Sany et al., “An overview of detection techniques for monitoring dioxin-like compounds: latest technique trends and their applications.” RSC Adv., 6, pp. 55415-55429, 2016). The cell contains a luciferase gene and its regulatory DNA. After the chemical substance to be tested binds to its corresponding receptor protein, the complex links to the regulatory DNA and induces expression of luciferase. Calux has been used to detect dioxin (Sany et al., 2016; Xu et al., “A rapid and reagent-free bioassay for the detection of dioxin-like compounds and other aryl hydrocarbon receptor (AhR) agonists using autobioluminescent yeast” Analytical and Bioanalytical Chemistry, 410, pp. 1247-1256, 2018), bisphenol A (Dusserre et al., “Using bisphenol A and its analogs to address the feasibility and usefulness of the CALUX-PPARγ assay to identify chemicals with obesogenic potential” Toxicology in Vitro, 53, pp. 208-221, 2018), heterocyclic aromatic amines (Steinberg et al., “Screening of molecular cell targets for carcinogenic heterocyclic aromatic amines by using calux reporter gene assays.” Cell Biology and Toxicology, 33, pp. 283-293, 2017) and other compounds. For dioxin, the detection takes approximately 24 hours (excluding sample preparation time), and the detection limit is 1 pM (Sany et al., 2016).

Avidin is a protein derived from both avians and amphibians that shows considerable affinity for biotin, a co-factor that plays a role in multiple eukaryotic biological processes. Avidin and other biotin-binding proteins, including streptavidin and neutravidin proteins, have the ability to bind up to four biotin molecules. The avidin-biotin complex is the strongest known non-covalent interaction (Kd=10⁻¹⁵ M) between a protein and a ligand. The bond formation between biotin and avidin is very rapid, and once formed, is unaffected by extremes of pH, temperature, organic solvents or other denaturing agents. These features of biotin and avidin—features that are shared by streptavidin and neutravidin—are useful for purifying or detecting proteins conjugated to either component of the interaction.

U.S. Pat. No. 11,275,082 discloses methods of detecting the presence of an analyte in a sample using recombinant spores or bacteria expressing recombinant proteins on the surface of the spores or the bacteria and detection systems using such recombinant spores or bacteria. The methods detect the presence of an analyte by detecting the binding of the recombinant proteins to the analyte using a signal-producing substance. An example of the method using LFA is shown in FIG. 2. Improving the detection limit of the above methods is desirable.

### SUMMARY OF THE INVENTION

In the present invention, the articles comprising microspheres, spores or bacteria conjugated to colloidal gold nanoparticles (AuNPs) are used as detecting agents in LFA. This improved detection method has several advantages, such as easier handling, lower cost, higher visibility, faster detection time, easier multiplexing, and lower detection limit/higher sensitivity.

Therefore, the present invention provides an article, a system and a method for detecting the presence of an analyte in a sample with improved limit of detection (LOD). In particular, the present invention provides an article for use in a system, such as a diagnostic kit, for detecting the presence of an analyte in a sample. The article comprises a microsphere, a spore or a bacterium conjugated to a high load of signal-producing substances, which are further conjugated to at least one binding agent capable of specifically bind to the analyte.

In one aspect, the signal-producing substance comprises dye, fluorescent dye, fluorescent protein, colloidal gold nanoparticles (AuNPs), nanoparticles with color, or enzymes capable of converting a substrate providing no signal to a substrate providing a signal. For example, the nanoparticles with color are dye-conjugated cellulose nanoparticles.

In one aspect, the limit of detection (LOD) of the analyte using the article, the system, or the method of the present invention ranges from 10⁻¹³ to 10⁻¹ mol. Preferably, the LOD is 10⁻¹⁴ or 10⁻¹⁵ mol.

In one aspect, the microsphere comprises polymer microsphere, silica microsphere or magnetic microsphere. Preferably, the polymer microsphere is a polystyrene microsphere.

In one aspect, the analyte is a protein, e.g., an antigen or an antibody. For example, if the analyte is a protein or an antigen, the agent capable of specifically bind to the analyte can be an antibody specific to the protein or antigen or a protein having affinity to the protein or antigen to be detected.

In one aspect, the protein expressed on the surface of spore or bacterium specifically binds to the signal-producing substance, which in turn specifically binds to the analyte. In one embodiment, the one or more affinity ligands of the microspheres or the proteins expressed on the surface of the sport or bacteria comprise a protein selected from streptavidin, avidin, an antibody, an antigen, protein A, protein G, protein L, and protein A/G, preferably streptavidin.

In another embodiment, the protein expressed on the surface of spore or bacterium is a fusion protein, wherein said fusion protein preferably comprises a coat protein of the spore and an exogenous protein or a membrane protein (or an artificial membrane protein) of the bacterium and an exogenous protein, wherein said exogenous protein is preferably streptavidin, avidin, protein A, protein G, protein L, or/and protein A/G.

In still another embodiment, the spore is produced by Bacillus species, preferably Bacillus subtilis, more preferably a strain of Bacillus subtilis selected from strains 168, PY79, W23 and NCIB3610. In a preferred embodiment, the fusion protein expressed by the spore comprises a coat protein selected from CotA, CotB, CotC, CotE, CotG, CotW, CotX, CotY and CotZ.

In one aspect, the bacteria are derived from Escherichia coli, Bacillus subtilis, Staphylococcal aureus, Staphylococcal xylosus, Staphylococcal carnosus, Neisseria gonorrhoeae, Salmonella enterica, Lactococcus lactis, or Streptococcus gordonii, preferably Escherichia coli.

In yet another aspect, the system further comprises a membrane with a test region and a control region, wherein antibodies, or antigens specific to the analyte are immobilized in the test region and/or the control region for detecting the presence or absence of the analyte in the sample. Preferably, said membrane is a nitrocellulose membrane commonly used in LFA.

In a preferred aspect, the binding of the microsphere or the protein expressed on the surface of spore or bacterium to the analyte is detected by LFA.

The present invention further provides a method of detecting an analyte in a sample using the above article and/or system.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows a diagram illustrating an example of conventional LFA systems.

FIG. 2 shows a diagram illustrating an example of the LFA system disclosed in U.S. Pat. No. 11,285,082.

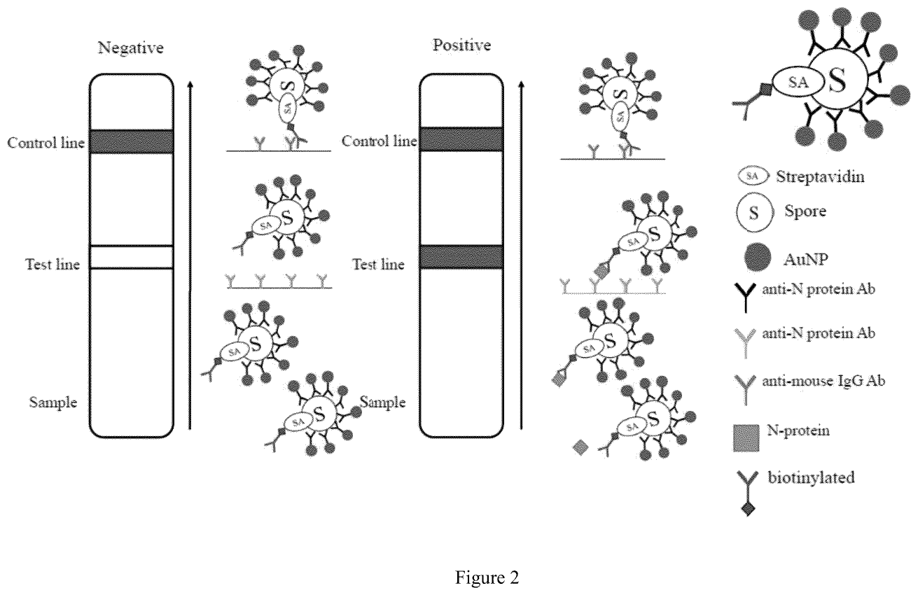

FIG. 3 shows a diagram illustrating an embodiment of the present invention for the detection of N protein by LFA using spores and gold nanoparticles (AuNPs).

FIG. 4 shows a diagram illustrating an embodiment of the present invention for the detection of N protein by LFA using E. coli and AuNPs.

FIG. 5 shows a diagram illustrating an embodiment of the present invention for the detection of N protein by LFA using streptavidin coated polystyrene microspheres and AuNPs.

FIG. 6 shows a diagram illustrating another embodiment of the present invention for the detection of anti-β-gal protein antibody by LFA using streptavidin coated polystyrene microspheres and AuNPs.

FIG. 7 shows the results of detection of N protein by LFA using spores and gold nanoparticles.

FIG. 8 shows the results of detection of N protein by LFA using E. coli and gold nanoparticles.

FIG. 9 shows the results of detection of N protein by LFA using streptavidin coated polystyrene microspheres and gold nanoparticles.

FIG. 10 shows the results of detection of N protein by conventional LFA using AuNPs.

### DETAILED DESCRIPTION

### Definitions

Unless defined otherwise, technical and scientific terms used herein have the same meaning as commonly understood by one of ordinary skill in the art to which this invention belongs.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

The term “analyte” as used herein refers to a substance to be detected for the presence or absence thereof in a sample, which can be a compound or a protein such as an antigen or an antibody.

The term “microsphere” as used herein refers to a microbead for diagnosis or research selected from (but not limited to) a polymer microsphere, silica microsphere or magnetic microsphere. For instance, the polymer microsphere is a polystyrene microsphere. The polystyrene microsphere comprises non-functionalized polystyrene, functionalized polystyrene, dyed polystyrene and affinity ligand coated polystyrene. For instance, the functionalized polystyrene comprises carboxylate-modified polystyrene, amine polystyrene, or other polystyrenes for the covalent immobilization of specific proteins, peptides, and nucleic acids. The dyed polystyrenes are impregnated with vibrant dyes for optimal visualization, which are frequently used in lateral flow assay. The affinity ligand coated polystyrene may be coated with recognition molecules, including (but not limited to) streptavidin, avidin, an antibody, an antigen, protein A, protein G, protein L, and protein A/G.

The term “bacterium” or “bacteria” refers to all Gram-positive and Gram-negative bacteria, including, but not limited to, Escherichia coli, Bacillus subtilis, Staphylococcal aureus, Staphylococcal xylosus, Staphylococcal carnosus, Neisseria gonorrhoeae, Salmonella enterica, Lactococcus lactis, and Streptococcus gordonii.

The term “spore” includes any spore commonly used to monitor sterilization processes and endospores. For example, spores from Bacillus subtilis, Bacillus circulans, Clostridium perfringens, Clostridium sporogenes or Bacillus stearothermophilus are useful. Spores from Bacillus subtilis strains 168, PY79, W23 and NCIB3610 are particularly useful. The spores can be unpurified or purified. For example, B. subtilis spores can be separated into heavy and light spores by differential centrifugation of an aqueous suspension. Heavy spores pellet after centrifugation for 12 to 15 minutes at 2,000×g, whereas the light spores remain in suspension. The light spores can be pelleted by centrifugation of the supernatant from the first centrifugation for 30 minutes at 2,000×g. The heavy spores can be further purified by filtering a dilute suspension of heavy spores through WhatmanGF/D glass fiber filters to remove bits of agar, denatured nucleoproteins and other debris that sediments with the spores in the first centrifugation.

The term “exogenous” as used herein means derived from outside the host strain, and the term “exogenous proteins” includes proteins, peptides, and polypeptides.

The “affinity ligand” of the affinity ligand coated polystyrene or the “protein” expressed by the bacterium or spore of the present invention comprises a protein selected from (but not limited to) streptavidin, avidin, an antibody, an antigen, protein A, protein G, protein L, and protein A/G.

The term “coat protein” is used herein in the broadest sense and includes any native protein present in the outer layer of spore coat and exposed on the spore surface, and functional fragments and functional amino acid sequence variants of such native proteins. The term includes native coat protein sequences of any spore-forming species and subspecies of the genus Bacillus, and functional fragments and functional amino acid sequence variants of such native coat protein sequences. The term “native” in this context is used to refer to native-sequence polypeptides, and does not refer to their origin or mode of preparation. Thus, native coat proteins may be isolated from their native source but can also be prepared by other means, e.g., synthetic and/or recombinant methods. Functional amino acid sequence variants include chimeric variants, comprising fusions of two or more native externally exposed spore coat protein sequences, or fragments thereof. Preferred coat proteins include CotA, CotB, CotC, CotE, CotG, CotW, CotX, CotY and CotZ.

The terms “spore coat protein B” and “CotB protein” are used interchangeably, and refer to externally exposed spore coat proteins that are characterized by a highly hydrophobic region at the C-terminus, and classified as CotB, such as CotB1 or CotB2 proteins based on sequence homologies. Preferably, the CotB proteins herein show significant amino acid sequence identity to each other and to the amino terminal two-thirds of the 42.9-kDa component of the B. subtilis spore coat associated with the outer coat layer. The sequence of a representative CotB protein herein is shown in SEQ ID NO: 1, which is specifically included within the definition of spore coat protein B (CotB) herein.

The terms “variant” and “amino acid sequence variant” are used interchangeably, and include substitution, deletion and/or insertion variants of native sequences. In a preferred embodiment, the protein variants have at least about 80% amino acid sequence identity, or at least about 85% amino acid sequence identity, or at least about 90% amino acid sequence identity, or at least about 92% amino acid sequence identity, or at least about 95% amino acid sequence identity, or at least about 95% amino acid sequence identity, or at least about 98% amino acid sequence identity with a native sequence.

A “functional” fragment or variant retains the ability to be propagated and stably displayed on the surface of a spore, such as a Bacillus spore.

The term “protein expression on the surface” is used herein in the broadest sense and includes complete and partial exposure of a protein, such as a spore coat protein or a recombinant protein.

The term “fusion” is used herein to refer to the combination of amino acid sequences of different origin in one polypeptide chain by in-frame combination of their coding nucleotide sequences. The term “fusion” explicitly encompasses internal fusions, i.e., insertion of sequences of different origin within a polypeptide chain, in addition to fusion to one of its termini.

The term “compound” refers to a chemical compound, such as biotin, dioxin, digoxin, and other protein-binding or antibody-binding chemicals.

The terms “polypeptide,” “peptide” and “protein” are used interchangeably herein to refer to a polymer of amino acid residues. The terms apply to amino acid polymers in which one or more amino acid residues are an artificial chemical analogue of a corresponding naturally occurring amino acid, as well as to naturally occurring amino acid polymers. Amino acids may be referred to herein by either their commonly known three letter symbols or the terminology recommended by Nomenclature Commission. Nucleotides, likewise, may be referred to by their commonly accepted single-letter codes, i.e., the one-letter symbols recommended by the IUPAC-IUB.

“Polynucleotide” and “nucleic acid” refer to a polymer composed of nucleotide units (ribonucleotides, deoxyribonucleotides, related naturally occurring structural variants, and synthetic non-naturally occurring analogs thereof) linked via phosphodiester bonds, related naturally occurring structural variants, and synthetic non-naturally occurring analogs thereof. Thus, the terms include nucleotide polymers in which the nucleotides and the linkages between them include non-naturally occurring synthetic analogs. It will be understood that, where required by context, when a nucleotide sequence is represented by a DNA sequence (i.e., A, T, G, C), this also includes an RNA sequence (i.e., A, U, G, C) in which “U” replaces “T.”

As used herein, an “antibody” refers to a protein comprising one or more polypeptides substantially or partially encoded by immunoglobulin genes or fragments of immunoglobulin genes. The term antibody is used to mean whole antibodies and binding fragments thereof. The recognized immunoglobulin genes include the kappa, lambda, alpha, gamma, delta, epsilon and mu constant region genes, as well as a myriad of immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. A typical immunoglobulin (e.g., antibody) structural unit comprises a tetramer. Each tetramer is composed of two identical pairs of polypeptide chains, each pair having one “light” (about 25 KDa) and one “heavy” chain (about 50-70 KDa). The N-terminus of each chain defines a variable region of about 100 to 110 or more amino acids primarily responsible for antigen recognition. The terms variable light chain (VL) and variable heavy chain (VH) refer to these light and heavy chains, respectively. In the present application, the term “antibody” specifically covers, without limitation, monoclonal antibodies, polyclonal antibodies, and antibody fragments.

An “antigen” refers generally to a substance capable of eliciting the formation of antibodies in a host or generating a specific population of lymphocytes reactive with that substance. Antigens may comprise macromolecules (e.g., polypeptides, proteins, and polysaccharides) that are foreign to the host.

The term “binding agent” refers to an agent that is conjugated to the signal-producing substance and specifically binds to the analyte. For example, the binding agent can be an antibody against the analyte or a binding partner of the analyte.

The terms “specific binding” and “specifically binds” when used in reference to the interaction of an antibody and a protein or peptide mean that the interaction is dependent upon the presence of a particular structure (i.e., for example, an antigenic determinant or epitope) on a protein; in other words, an antibody is recognizing and binding to a specific protein structure rather than to proteins in general. For example, if an antibody is specific for epitope “A,” the presence of a protein containing epitope A (or free, unlabeled A) in a reaction containing labeled “A” and the antibody will reduce the amount of labeled A bound to the antibody.

The term “detecting agent” refers to an agent that can bind the analyte and produce a signal for detection. Preferably, the detecting agent is a spore, bacterial or microsphere, conjugated to modify AuNPs comprising a binding partner to the analyte. More preferably, the detecting agent includes a primary antibody specific for the analyte to be detected and a secondary antibody specific for the primary antibody. In one embodiment, the detecting agent comprises a signal-producing substance. Such detection may be performed with techniques commonly known in the art, including, but not limited to, flow cytometry, lateral flow assay, and ELISA.

The term “signal-producing substance” refers to a substance providing a signal that can be detected by eye or detectors such as a fluorescence microscope. Such signal-producing substance includes, but is not limited to, a dye, fluorescent dye, fluorescent protein, colloidal gold nanoparticles, nanoparticles with color, cellulose nanoparticles, or enzymes capable of converting a substrate providing no signal to a substrate providing a signal. The signal-producing substance may be a detecting agent for detecting the binding of the protein or binding agent to the analyte.

The term “label” or “labeled with” is used herein to refer to any composition detectable by spectroscopic, photochemical, biochemical, immunochemical, electrical, optical or chemical means. Such labels include biotin for staining with labeled streptavidin conjugate, magnetic beads (e.g., Dynabeads), fluorescent dyes (e.g., fluorescein, Texas Red, rhodamine, green fluorescent protein, and the like), radiolabels (e.g., 3 H, 125 1, 35 S, 14 C, or 32 P), enzymes (e.g., horse radish peroxidase (HRP), alkaline phosphatase and others commonly used in an ELISA), calorimetric labels such as colloidal gold nanoparticles or colored glass or plastic (e.g., polystyrene, polypropylene, latex, etc.) beads, and colorimetric labels such as nanoparticles with color or cellulose nanoparticles. Patents teaching the use of such labels include, but are not limited to, U.S. Pat. Nos. 3,817,837; 3,850,752; 3,939,350; 3,996,345; 4,277,437; 4,275,149; and 4,366,241 (all herein incorporated by reference). The labels contemplated in the present invention may be detected by many methods. For example, radiolabels may be detected using photographic film or scintillation counters, and fluorescent markers may be detected using a photodetector to detect emitted light. Enzymatic labels are typically detected by providing the enzyme with a substrate and detecting the reaction product produced by the action of the enzyme on the substrate, and calorimetric labels are detected by simply visualizing the colored label.

The term “lateral flow assay” refers to an assay in which detection is based on the specificity and immunoaffinity between an antigen and an antibody, using colloidal gold as a color developing agent. In an example of such assay, an antibody against the target substance is first immobilized in the test line on a nitrified cellulose membrane, and a secondary antibody against the above antibody against the target substance is immobilized in the control line on the membrane; a colloidal gold particle and another antibody against the target substance are then conjugated and immobilized on a conjugated release pad. When a sample contains the target substance, the colloidal gold-antibody against the target substance conjugate on the conjugated release pad will bind the target substance, and once the complex reaches the test line, the antibody against the target substance in the test line will bind the above complex and lead to color development. The secondary antibody immobilized in the control line can bind the colloidal gold-antibody against the target substance conjugate and serves as a positive control. In the present invention, antibodies, antigens or competing agents may be immobilized in the positive region and/or the negative region for detecting the presence or absence of an analyte in the sample. For example, one or more substances selected from the following may be immobilized in the positive or negative region on the membrane: antibodies against the analyte, the carrier protein, the spore, the protein on the spore or the binding agent; competing agents; and antigens recognized by the analyte.

The term “N protein” refers to a nucleoprotein comprising a full-length SARS-CoV-2 nucleocapsid protein. Preferably, the N protein is the commercialized product HyTest Cat. #8COV3. N protein is an antigen for detection of anti-(SARS-CoV-2 nucleocapsid protein) antibodies.

### Detection by Lateral Flow Assay

The detection system of the present invention can be used to detect the presence of an analyte in a sample through lateral flow assay using a detecting agent labeled with dyes, fluorescent dyes, fluorescent proteins, colloidal gold nanoparticles, nanoparticles with color, cellulose nanoparticles, or enzymes which can convert a substance providing no signal to a substance providing signal.

Preferably, the detecting agent is labeled with gold nanoparticles. In one embodiment, the gold nanoparticles are conjugated with an anti-spore antibody and an antibody specific to the analyte to form an AuNP-Ab complex. In one embodiment, the gold nanoparticles are conjugated with an anti-E. coli antibody and an antibody specific to the analyte to form an AuNP-Ab complex. In another embodiment, the gold nanoparticles are conjugated with an anti-streptavidin antibody and an antibody specific to the analyte to form an AuNP-Ab complex. In a further embodiment, the gold nanoparticles are conjugated with an anti-streptavidin antibody and an affinity ligand specific to the analyte to form an AuNP-ligand complex. In one embodiment, the detecting agent is a spore conjugated to an AuNP-Ab complex or AuNP-ligand complex. In another embodiment, the detecting agent is a E. coli conjugated to an AuNP-Ab complex or AuNP-ligand complex. Preferably, the sport or E. coli expresses a recombinant fusion protein, wherein said fusion protein preferably comprises a coat protein of the spore and an exogenous protein or a membrane protein (or an artificial membrane protein) of the recombinant bacterium and an exogenous protein, wherein said exogenous protein is preferably streptavidin, avidin, protein A, protein G, protein L, or/and protein A/G. In still another embodiment, the spore is produced by Bacillus species, preferably Bacillus subtilis, more preferably a strain of Bacillus subtilis selected from strains 168, PY79, W23 and NCIB3610. In a preferred embodiment, the fusion protein expressed by the spore comprises a coat protein selected from CotA, CotB, CotC, CotE, CotG, CotW, CotX, CotY and CotZ.

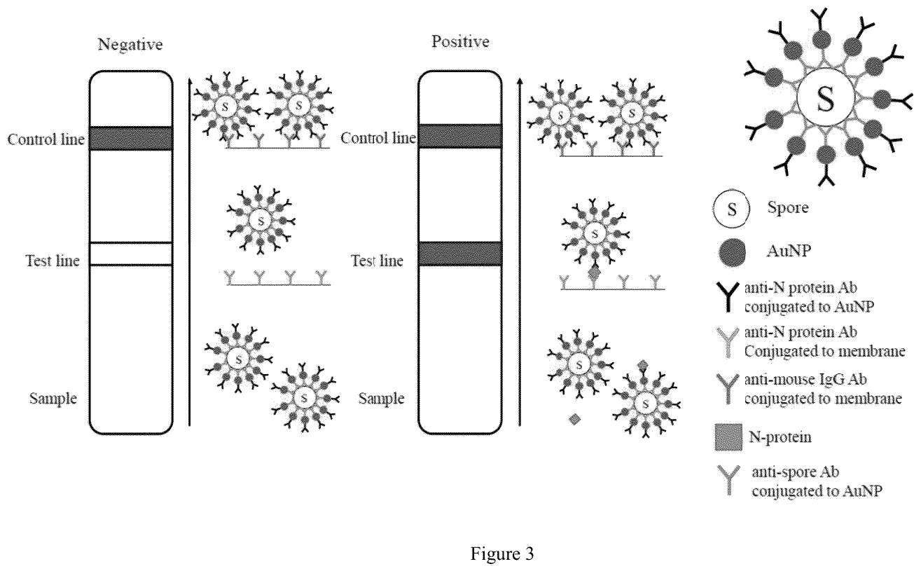

For example, please refer to the embodiment wherein detecting agents are spores conjugated to colloidal gold nanoparticles (AuNPs) as shown in FIG. 3. In this embodiment, the analyte is N protein and the article for detecting the presence of N protein is a spore expressing one or more protein on the surface thereof conjugated to a high load of signal producing substances, i.e., AuNPs, through an antibody specific to the protein expressed on the surface of the spore. In this embodiment, the binding agents is anti-N protein antibody conjugated to the AuNPs, and the protein expressed on the surface of the spore is streptavidin.

In the above embodiment, the membrane of the LFA system herewith comprises the control line dispensed with anti-mouse IgG secondary antibody and the test line dispensed with anti-N protein antibody. When the sample includes N protein, the membrane would show two positive lines, the control line and the test line. In this scenario, N protein in the sample would concurrently bind to the anti-N protein antibody conjugated with the AuNP and the anti-N protein antibody fixed on the membrane. Therefore, the signals produced by the detecting agents (i.e., the AuNP-Ab complex conjugated to spores) shown at the test line indicate the presence of N protein in the test sample. On the other hand, when the sample lacks N protein, the membrane would show only one line, the control line.

In one embodiment, the detecting agents are bacteria conjugated to colloidal gold nanoparticles (AuNPs). Preferably, the bacteria are derived from Escherichia coli, Bacillus subtilis, Staphylococcal aureus, Staphylococcal xylosus, Staphylococcal carnosus, Neisseria gonorrhoeae, Salmonella enterica, Lactococcus lactis, or Streptococcus gordonii, preferably Escherichia coli.

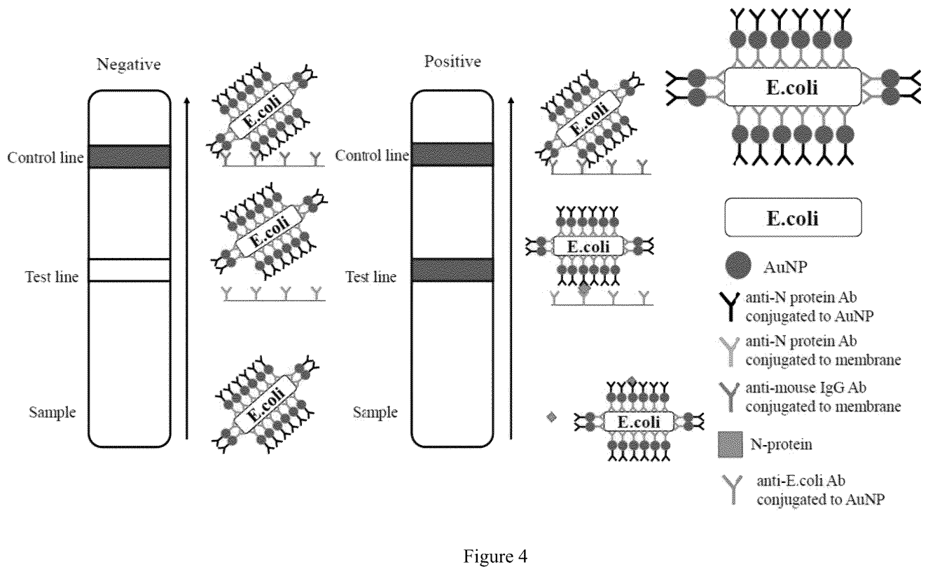

For example, please refer to the embodiment wherein detecting agents are bacteria conjugated to AuNPs as shown in FIG. 4. In this embodiment, the analyte is N protein and the article for detecting the presence of N protein is E. coli expressing one or more proteins on the surface thereof conjugated to a high load of AuNPs through an antibody specific to the protein expressed on the surface of E. coli. In this embodiment, the binding agent is an anti-N protein antibody conjugated to the AuNPs and the protein expressed on the surface of the bacteria is streptavidin. The membrane of the LFA system herewith comprises the control line dispensed with anti-mouse IgG secondary antibody and the test line dispensed with anti-N protein antibody. When the sample includes N protein, the membrane would show two lines, the control line and the test line. In this scenario, N protein in the sample would concurrently bind to the anti-N protein antibody conjugated with the AuNP and the anti-N protein antibody fixed on the membrane. Therefore, the signals produced by the detecting agents (i.e., the AuNP-Ab complex conjugated to E. coli) at the test line indicate the presence of N protein in the test sample. On the other hand, when the sample lacks N protein, the membrane would show only one line, the control line.

In one embodiment, the detecting agent is a microsphere conjugated to colloidal gold nanoparticles (AuNPs). Preferably, the microspheres are polystyrene microspheres. Specifically, the polystyrene microspheres are coated with affinity ligands for conjugation with the AuNP-Ab complex.

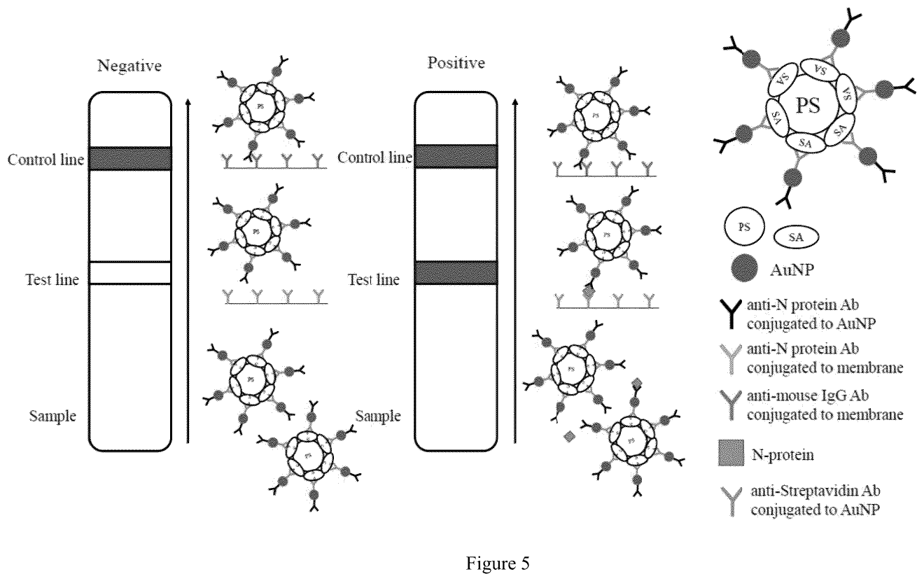

For example, please refer to the embodiment wherein detecting agents are microspheres conjugated to colloidal gold nanoparticles (AuNPs) as shown in FIG. 5. In this embodiment, the analyte is N protein and the article for detecting the presence of N protein is a microsphere coated with streptavidin conjugated to the AuNP through an anti-streptavidin antibody. In this embodiment, the binding agent is anti-N protein antibody conjugated to the AuNPs. The membrane of the LFA system herewith comprises the control line dispensed with anti-mouse IgG secondary antibody and the test line dispensed with anti-N protein antibody. When the sample includes N protein, the membrane would show two positive lines, the control line and the test line. In this scenario, N protein in the sample would concurrently bind to the anti-N protein antibody conjugated with the AuNP and the anti-N protein antibody fixed on the membrane. Therefore, the signals produced by the detecting agents (i.e., the AuNP-Ab complex conjugated to the microsphere) at the test line indicate the presence of N protein in the test sample. On the other hand, when the sample lacks N protein, the membrane would show only one line, the control line.

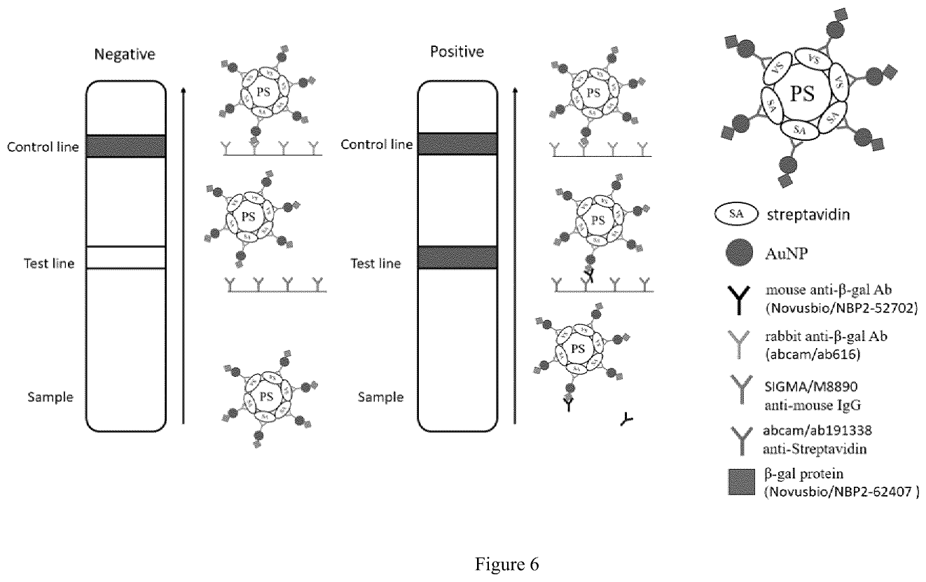

For another example, please refer to the embodiment wherein detecting agents are microspheres conjugated to colloidal gold nanoparticles (AuNPs) as shown in FIG. 6. In this embodiment, the analyte is mouse anti-β-gal antibody and the articles for detecting the presence of mouse anti-β-gal antibody are streptavidin coated polystyrene microspheres. In this embodiment, the binding agent is β-gal protein conjugated to the AuNPs. The membrane of the LFA system herewith comprises the control line dispensed with rabbit anti-β-gal antibody and the test line dispensed with anti-mouse IgG antibody. When the sample includes mouse anti-β-gal antibodies, the membrane would show two positive lines, the control line and the test line. In this scenario, different from the previous embodiment, such analyte (i.e., mouse anti-β-gal antibodies) would concurrently bind to the β-gal protein conjugated to AuNP and the anti-mouse IgG Ab fixed on the membrane. Therefore, the signals produced from the detecting agents (i.e., the AuNP-β-gal protein complex conjugated to the microspheres) at the test line indicate the presence of anti-β-gal antibody in the test sample. On the other hand, when the sample lacks anti-β-gal antibody, the membrane would show only one line, the control line.

According to the present invention, the limit of detection (LOD) of using the detecting agent of the invention had a decrease of 10 to 1000 times in comparison with the use of only colloidal gold nanoparticles. In one embodiment, the LOD of the analyte is 10⁻¹⁴ mol. In another embodiment, the LOD of the analyte is 10⁻¹⁵ mol. Since the cellulose nanoparticle has superior visibility, it enables better detection of low antigen concentration than conventional labels. Higher sensitivity means fewer false negatives due to antigen concentration being below the LOD. Accordingly, early diagnosis may be established. Furthermore, higher sensitivity enables reduced physical discomfort for patients through the use of saliva or other low-concentration samples (e.g., nasal swabs or self-blow nasal discharge specimens) rather than throat swabs or blood samples.

Further details of the invention are illustrated by the following non-limiting examples.

### Example 1: Detection of N Protein by LFA Using Spores and Gold Nanoparticles (AuNPs) as Shown in FIG. 3

1.1 AuNP Modification and Coating Spore with Modified AuNP

16 μl of potassium carbonate solution at a concentration of 26 mM was added to 100 μl of AuNP solution (Taiwan Advanced Nanotech/Nano Gold-40). 0.5 μl of anti-N protein Ab (HyTest Cat. #3CV4/C524) at a concentration of 1 mg/ml and 0.5 μl of anti-spore antibody (Mybiosource/mbs612878) at a concentration of 1 mg/ml were added to the AuNP solution and the mixture was reacted at 4° C. for 16 hours, followed by addition of 16 μl of 10% BSA and waiting for 30 minutes. The mixture was then centrifuged under 4000×g at 4° C. for 40 minutes, and the supernatant was removed. The precipitate was resuspended with 100 μl of PBS. 1 ml of spores (B. subtilis 168/BCRC number: 17890) with OD₆₀₀=1 obtained in 1-4 of Example 1 was centrifuged under 12000×g at 4° C. for 3 minutes, and the supernatant was removed. The precipitate was mixed thoroughly with the resuspended AuNP in PBS so that the modified AuNP was conjugated to the spore, and the mixture (AuNP-spore conjugate) was stored at 4° C. for use.

### 1.2 Strip Preparation

1 mg/ml of anti-mouse IgG secondary antibody (SIGMA/M8890) and 1 mg/ml of anti-N protein antibody (HyTest Cat. #3CV4/C706) were dispensed to the control line and the test line, respectively, on the membrane (Millipore/HF1200) (2 μl per cm on the membrane), followed by a drying step at room temperature for 2 hours. The membrane was then immersed in 1% PVA (polyvinyl alcohol) solution for 30 minutes, followed by a drying step at room temperature for 2 hours. After being dried, the membrane was cut into strips with a width of 0.5 cm and stored at 4° C.

### 1.3 Detection

100 μl of the AuNP-spore conjugate prepared in 2-1 above was mixed thoroughly with 1 μl of N-protein (HyTest Cat. #8COV3) at different concentrations. Afterward, the strip was immediately placed in the above solution mixture, and a photograph was taken to record the result after drying for about 20 minutes.

### 1.4 Result

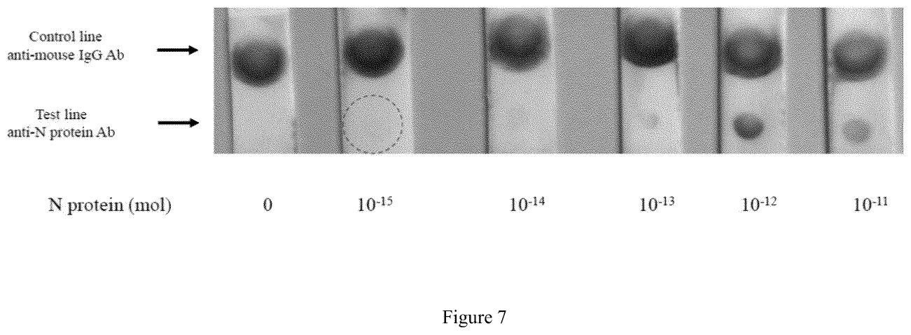

It was found that when the amount of the N protein is 10⁻¹⁵ mol, the test line shows a signal, and the greater the antibody amount is, the stronger the signal is (see FIG. 7).

### Example 2: Detection of N Protein by LFA Using E. coli and Gold Nanoparticles (AuNPs) as Shown in FIG. 4

2.1 AuNP Modification and Coating E. coli with the Modified AuNP

100 μl of AuNP solution (Taiwan Advanced Nanotech/Nano Gold-40) was added to 16 μl of potassium carbonate solution at a concentration of 26 mM. 0.5 μl of anti-N protein Ab (HyTest Cat. #3CV4/C524) at a concentration of 1 mg/ml and 0.5 μl of anti-E. coli antibody (abcam/ab137967) (from rabbit) at a concentration of 1 mg/ml were added to the AuNP solution, and the mixture was reacted at 4° C., 450 rpm for 16 hours, followed by addition of 16 μl of 10% BSA and waiting at 4° C., 450 rpm for 30 minutes. The mixture was then centrifuged under 4000×g at 4° C. for 40 minutes, and the supernatant was removed. The precipitate was resuspended with 100 μl of PBS. 1 ml of the cultured E. coli (Escherichia coli str. K-12 substr. MG1655/BCRC number: 51956) was centrifuged under 12000×g at 4° C. for 3 minutes, and the supernatant was removed. The precipitate was added to the previous mixture and was mixed thoroughly with the resuspended AuNP in PBS so that the modified AuNP was conjugated to the E. coli, and the mixture (AuNP-E. coli conjugate) was stored at 4° C. for use.

### 2.2 Strip Preparation

Mount the nitrocellulose membrane (MILLIPORE Cat. #SHF1200425) on the backing card. 2 mg/ml of anti-mouse IgG secondary antibody (SIGMA/M8890) and 1 mg/ml of anti-N protein antibody (HyTest Cat. #3CV4/C706) were respectively dispensed to the control line and the test line on the membrane (MILLIPORE Cat. #SHF1200425) (2 μl per cm on the membrane), followed by a drying step at room temperature for 2 hours. The membrane was then immersed in 1% PVA (polyvinyl alcohol) solution for 30 minutes, followed by a drying step at room temperature for 2 hours. After being dried, the membrane was cut into strips with a width of 0.5 cm and stored at 4° C.

### 2.3 Detection

100 μl of the AuNP-E. coli conjugate prepared in 3-1 above was mixed thoroughly with 1 μl of N-protein (HyTest Cat. #8COV3) at different concentrations. Afterward, the strip was immediately placed in the above solution mixture, and a photograph was taken to record the result after drying for about 20 minutes.

### 2.4 Result

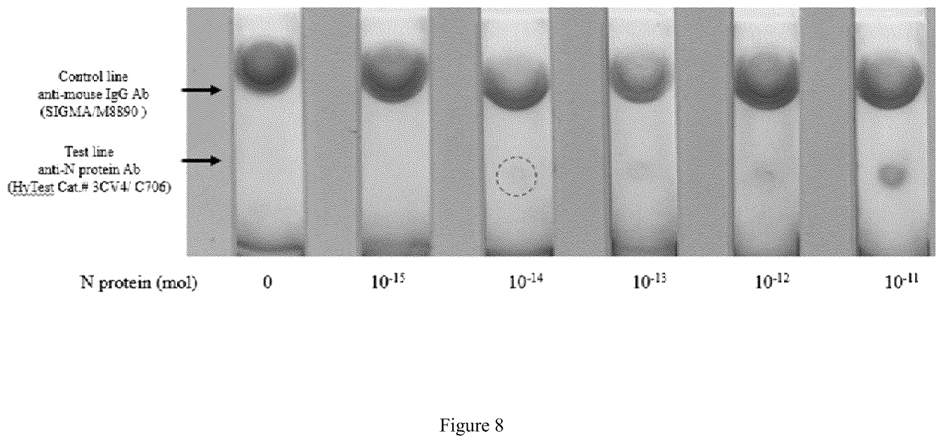

It was found that when the amount of the N protein is 10⁻¹⁴ mol, the test line shows a signal, and the greater the antibody amount is, the stronger the signal is (see FIG. 8).

### Example 3: Detection of N Protein by LFA Using Streptavidin Coated Polystyrene Microsphere and Gold Nanoparticles (AuNPs) as Shown in FIG. 5

3.1 AuNP Modification and Coating Polystyrene Microsphere with the Modified AuNP

100 μl of AuNP solution (Taiwan Advanced Nanotech/Nano Gold-40) was added to 16 μl of potassium carbonate solution at a concentration of 26 mM. 0.5 μl of anti-N protein Ab (HyTest Cat. #3CV4/C524) at a concentration of 1 mg/ml and 0.5 μl of anti-streptavidin Ab (abcam/ab191338) at a concentration of 1 mg/ml were added to the AuNP solution, and the mixture was reacted at 4° C. for 16 hours, followed by addition of 16 μl of 10% BSA and waiting for 30 minutes. The mixture was then centrifuged under 4000×g at 4° C. for 40 minutes, and the supernatant was removed. The precipitate was resuspended with 100 μl of PBS and mixed thoroughly with 2 μl of streptavidin coated polystyrene microsphere (Bangs Laboratories Cat. #CP01004/CP01N, 1.557×10¹⁰ microspheres/ml). The mixture (AuNP-microsphere conjugate) was then stored at 4° C. for use.

### 3.2 Strip Preparation

1 μL of 2 mg/ml of anti-mouse IgG secondary antibody (SIGMA/M8890) and 1 μL of 1 mg/ml of anti-N protein Ab (HyTest Cat. #3CV4/C706) were dispensed to the control line and the test line, respectively, on the membrane (Millipore/HF1200) (2 μl per cm on the membrane), followed by a drying step at drying cabinet for 2 hours. The membrane was then immersed in 1% PVA for 30 minutes followed by a drying step at drying cabinet for 2 hours. After being dried, the membrane was cut into strips with a width of 0.5 and stored at 4° C.

### 3.3 Detection

100 μl of the AuNP-microsphere conjugate mixture prepared in 3-1 above was mixed thoroughly with 1 μl of N-protein (HyTest Cat. #8COV3) at different concentrations. Afterward, the strip was immediately placed in the above solution mixture, and a photograph was taken to record the result after drying for about 20 minutes.

### 3.4 Result

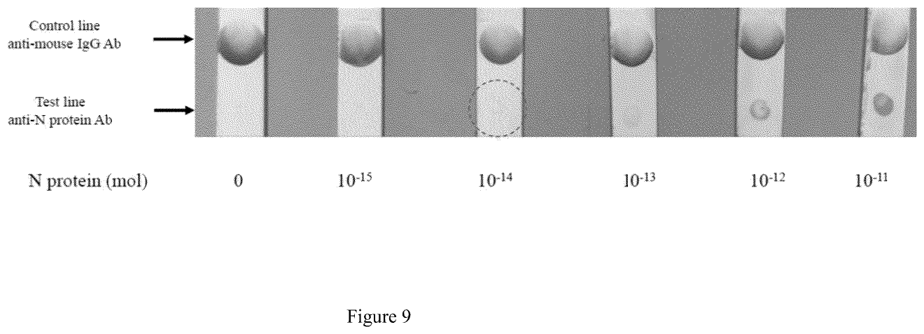

It was found that when the amount of the N protein is 10⁻¹⁴ mol, the positive line shows a signal, and the greater the antibody amount is, the stronger the signal is (see FIG. 9).

### Example 4: Detection of Anti-β-Gal Protein Antibody by LFA Using Streptavidin Coated Polystyrene Microsphere and Gold Nanoparticles (AuNPs) as Shown in FIG. 6

4.1 AuNP Modification and Coating Polystyrene Microsphere with the Modified AuNP

100 μl of AuNP solution (Taiwan Advanced Nanotech/Nano Gold-40) was added to 16 μl of potassium carbonate solution at a concentration of 26 mM. 0.5 μl of β-gal protein (Novusbio/NBP2-62407) at a concentration of 1 mg/ml and 0.5 μl of anti-streptavidin Ab (abcam/ab191338) at a concentration of 1 mg/ml were added to the AuNP solution and the mixture was reacted at 4° C. for 16 hours with 450 rpm, followed by the addition of 16 μl of 10% BSA and waiting for 30 minutes. The mixture was then centrifuged under 4000×g at 4° C. for 40 minutes, and the supernatant was removed. The precipitate was resuspended with 100 μl of PBS and mixed thoroughly with 2 μl of streptavidin-coated polystyrene microsphere (Bangs Laboratories Cat. #CP01004/CP01N, 1.557×10¹⁰ microspheres/ml), and the mixture (AuNP-microsphere conjugate) was stored at 4° C. for use.

### 4.2 Strip Preparation

1 μL of 2 mg/ml of anti-mouse IgG secondary antibody (SIGMA/M8890) and 1 μL of 1 mg/ml of rabbit anti-β-gal Ab (abcam/ab616) were dispensed to the test line and the control line, respectively, on the membrane (Millipore/HF1200) (2 μl per cm on the membrane), followed by a drying step at drying cabinet for 2 hours. The membrane was then immersed in 1% PVA for 30 minutes followed by a drying step at drying cabinet for 2 hours. After being dried, the membrane was cut into strips with a width of 0.5 and stored at 4° C.

### 4.3 Detection

100 μL of the AuNP-microsphere conjugate prepared in 4.1 above was mixed thoroughly with 1 μl of mouse anti-β-gal Ab (Novusbio/NBP2-52702) at different concentrations. Afterward, the membrane was immediately placed in the above solution mixture, and a photograph was taken to record the result after drying for about 20 minutes.

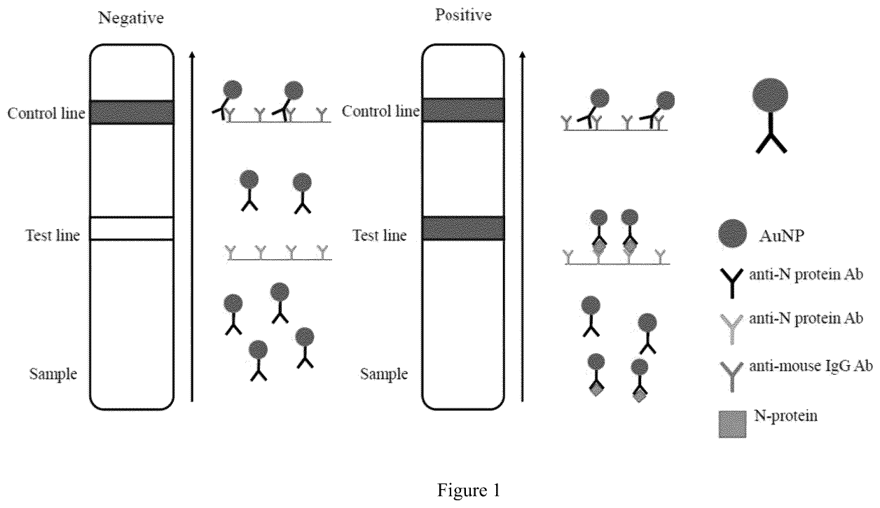

### Example 5: Detection of N-Protein by Conventional LFA Using Gold Nanoparticles (AuNPs) as Shown in FIG. 1

### 5.1 AuNP Modification

100 μl of AuNP solution (Taiwan Advanced Nanotech/Nano Gold-40) was added to 16 μl of potassium carbonate solution at a concentration of 26 mM. 1 μl of anti-N protein antibody (HyTest Cat. #3CV4/C524) at a concentration of 1 mg/ml was added to the AuNP solution, and the mixture was reacted at 4° C. for 16 hours, followed by addition of 16 μl of 10% BSA and waiting for 30 minutes. The mixture was then centrifuged under 4000×g at 4° C. for 40 minutes. Afterward, the supernatant was removed, and the precipitate was resuspended with 100 μl of PBS and stored at 4° C. for use.

### 5.2 Strip Preparation

1 mg/ml of anti-N protein antibody (HyTest Cat. #3CV4/C706) and 2 mg/mL of anti-mouse IgG Ab (SIGMA/M8890) were dispensed to the test line and the control line, respectively, on the membrane (Millipore/HF1200) (2 μl per cm on the membrane), followed by a drying step at room temperature for 2 hours. The membrane was then immersed in 1% PVA for 30 minutes followed by a drying step at room temperature for 2 hours. Afterward, the membrane was immersed in 5% sucrose solution for 30 seconds. After being dried, the membrane was cut into strips with a width of 0.5 cm and stored at 4° C.

### 5.3 Detection

The modified AuNP was mixed thoroughly with 1 μl of N protein at different concentrations. Afterward, the strip was immediately placed in the above solution mixture, and a photograph was taken to record the result after drying for about 10 minutes.

### 5.4 Result

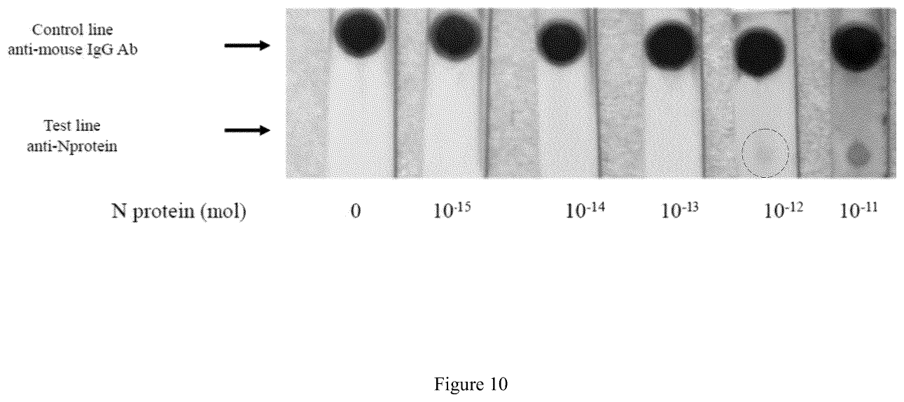

It was found that when the amount of N protein is 10⁻¹² mol, the positive line shows a faint signal, and the higher the N protein concentration is, the stronger the signal is (see FIG. 10). Furthermore, there is no signal in the positive line for the samples containing N protein of 10⁻¹³ mol, 10⁻¹⁴ mol, or 10⁻¹⁵ mol.

Clearly, the sensitivity of the detection system of the present invention with respect to detection of N protein by LFA using the conjugates as demonstrated in Examples 1 to 4 is increased 1,000-fold compared with conventional LFA methods.

1. An article for detecting the presence of an analyte in a sample, comprising (i) a microsphere coated with an affinity ligand, or a spore or bacterium expressing one or more proteins on the surface thereof, (ii) a signal-producing substances, and (iii) a binding agent, wherein the signal-producing substance is conjugated with the binding agent and an antibody specific to the affinity ligand on the microsphere or the protein expressed by the spore or bacterium, wherein the signal-producing substances are conjugated to the microsphere, spore or bacterium through the binding of the antibodies specific to the affinity ligand or the protein.

2. The article of claim 1, wherein the spore is produced by Bacillus species.

3. The article of claim 2, wherein the spore is produced by a strain of Bacillus subtilis selected from strains BH60, 168, PY79, W23 and NCIB3610.

4. The article of claim 1, wherein the bacterium is derived from Escherichia coli, Bacillus subtilis, Staphylococcal aureus, Staphylococcal xylosus, Staphylococcal carnosus, Neisseria gonorrhoeae, Salmonella enterica, Lactococcus lactis, or Streptococcus gordonii.

5. The article of claim 4, wherein the bacterium is derived from Escherichia coli.

6. The article of claim 1, wherein the protein expressed on the surface of the spore or bacterium is a fusion protein.

7. The article of claim 6, wherein the fusion protein comprises (i) a coat protein of the spore and an exogenous protein or (ii) a membrane protein of the bacterium and an exogenous protein.

8. The article of claim 7, wherein the exogenous protein is streptavidin, avidin, protein A, protein G, protein L, and/or protein A/G.

9. The article of claim 6, wherein the fusion protein is expressed by the spore and comprises a coat protein selected from CotA, CotB, CotC, CotE, CotG, CotW, CotX, CotY and CotZ.

10. The article of claim 1, wherein the microsphere comprises a polymer microsphere, silica microsphere or magnetic microsphere.

11. The article of claim 10, wherein the polymer microsphere is a polystyrene microsphere.

12. The article of claim 11, wherein the polystyrene microsphere comprises non-functionalized polystyrene, functionalized polystyrene, dyed polystyrene, and affinity ligand coated polystyrene.

13. The article of claim 12, wherein affinity ligand is a protein selected from streptavidin, avidin, an antibody, an antigen, protein A, protein G, protein L, and protein A/G.

14. The article of claim 13, wherein the affinity ligand is streptavidin.

15. The article of claim 1, wherein the binding agent is an antibody against the analyte.

16. The article of claim 1, wherein the signal-producing substance comprises dye, fluorescent dye, fluorescent protein, colloidal gold nanoparticles, nanoparticles with color, or enzymes capable of converting a substrate providing no signal to a substrate providing a signal.

17. A system for detecting the presence of an analyte in a sample comprising the article of claim 1.

18. The system of claim 17, which is for used in LFA.

19. The system of claim 18, wherein the system further comprises a membrane with a test region and a control region, wherein (a) when the analyte is an antigen, an antibody specific to the analyte is immobilized in the test region, and a secondary antibody against the antibody specific to the analyte is immobilized in the control region, or (b) when the analyte is an antibody, a secondary antibody against the analyte is immobilized in the test region, and an antibody specific to an antigen recognized by the analyte is immobilized in the control region.

20. The system of claim 18, which is a diagnostic kit.

21. The system of claim 18, wherein the membrane is a nitrocellulose membrane.

22. A method of detecting the presence of an analyte in a sample using the system of claim 17.
